(19) United States
(12) Patent Application Publication
(10) Pub. No.: US 2026/0248460 A1
(43) Pub. Date: Aug. 27, 2026

(54) INFORMATION PROCESSING APPARATUS, DIAGNOSTIC APPARATUS, MEDICAL SYSTEM, AND STORAGE MEDIUM

(71) Applicant: Konica Minolta, Inc., Tokyo (JP)

(72) Inventors: Hidekazu Takahashi, Toyonaka-shi (JP); Hisashi Wada, Tokyo (JP); Kohei Ikeda, Nishitokyo-shi (JP)

(73) Assignee: Konica Minolta, Inc., Tokyo (JP)

(21) Appl. No.: 19/548,962

(22) Filed: Feb. 25, 2026

(30) Foreign Application Priority Data
Feb. 27, 2025 (JP) 2025-029932

(51) Int. Cl. A61B 5/00

(52) U.S. Cl. CPC A61B 5/7271; A61B 5/742

(57) ABSTRACT

An information processing apparatus includes, a first hardware processor that controls imaging of a medical image and that generates first diagnostic assistance information by performing predetermined computer processing on the medical image; a first display that displays at least the medical image; and an inputter for inputting second diagnostic assistance information regarding the medical image, wherein the hardware processor outputs the first diagnostic assistance information and the second diagnostic assistance information to an external device in a distinguishable manner.

### CROSS-REFERENCE TO RELATED APPLICATION

The entire disclosure of Japanese Patent Application No. 2025-029932, filed on Feb. 27, 2025, including description, claims, drawings and abstract is incorporated herein by reference.

### BACKGROUND OF THE INVENTION

### Technical Field

The present disclosure relates to an information processing apparatus, a diagnostic apparatus, a medical system, and a storage medium.

### Description of Related Art

In recent years, in order to reduce a burden on a doctor, a STAT image report has attracted attention, in which a medical radiology technician promptly conveys an image with a finding of a disease related to vital prognosis and needing urgent attention to the doctor (clinician) after imaging. According to the STAT image report, a captured image is immediately reported to the doctor, so that quick and appropriate treatment can be performed.

For example, according to Japanese U.S. Patent No. 7405546, when a life-threatening disease is detected from a medical image obtained by imaging a patient, contact information of the doctor, availability status of the doctor at the hospital or the like are acquired. Furthermore, the above patent describes an information processing apparatus which provides a notification of information on a doctor or the like who can be contacted together with a detection result of the disease.

In addition, in recent years, a computer-aided detection/diagnosis (CAD) system has been put into practical use, and according to such system, the medical image is analyzed by using a computer, a lesion detection area detected by an image analysis is presented to the doctor, and the doctor is asked for his/her determination. In diagnostic assistance using such CAD, diagnostic assistance information is generated by performing computer processing, such as artificial intelligence (AI) analysis, on the medical image, and is provided to the doctor.

When diagnosis support by the CAD is performed, the diagnostic assistance information by the CAD and the diagnostic assistance information by the technician coexist for one medical image. In this case, the doctor may confuse the two.

For example, in a case where the information of the CAD by the AI analysis or the like includes comments or suggestions with sentences like the technician, it is difficult to distinguish such information by the AI analysis from the information by the technician. When the CAD information uses a human-like fine expression, it is more difficult to distinguish the information.

On the contrary, there is a case where the technician attaches only an annotation and does not attach the sentence such as the comment. In a case where the technician performs mechanical processing like CAD, it may be more difficult to distinguish between the two.

In this respect, the apparatus described in Japanese Patent No. 7405546 can contact the doctor or the like at an early stage when the disease needing urgent attention is detected. However, it is not possible to provide diagnostic assistance information for the medical image by distinguishing whether the diagnostic assistance information is provided by the technician or the CAD.

### SUMMARY OF THE INVENTION

The present disclosure has been made in view of the above-described problems in the related art, and an object of the present disclosure is to provide a doctor with diagnostic assistance information obtained by computer processing and diagnosis assistance information by a technician in an easily distinguishable manner.

According to one aspect of the present disclosure, an information processing apparatus reflecting one aspect of the present disclosure includes,
a first hardware processor that controls imaging of a medical image and that generates first diagnostic assistance information by performing predetermined computer processing on the medical image; a first display that displays at least the medical image; and
an inputter for inputting second diagnostic assistance information regarding the medical image,
wherein the hardware processor outputs the first diagnostic assistance information and the second diagnostic assistance information to an external device in a distinguishable manner.

According to another aspect of the present disclosure, a diagnostic apparatus includes:
a second hardware processor that acquires first diagnostic assistance information generated by performing predetermined computer processing on a medical image, and second diagnostic assistance information that is information input regarding the medical image and that is information distinguishable from the first diagnostic assistance information; and
a second display that displays at least the medical image.

According to another aspect of the present disclosure, a medical system including the above-described information processing apparatus, the medical system including:
a diagnostic apparatus including, a second hardware processor that acquires the first diagnostic assistance information and the second diagnostic assistance information, and a second display that displays at least the medical image.

According to another aspect of the present disclosure, a non-transitory computer readable storage medium storing a program that causes a computer of an apparatus including a display to,
control capturing of a medical image,
cause the display to display at least the medical image,
generate first diagnostic assistance information by performing predetermined computer processing on the medical image,
accept input of second diagnostic assistance information on the medical image, and
output the first diagnostic assistance information and the second diagnostic assistance information to the external device in a distinguishable manner.

### BRIEF DESCRIPTION OF THE DRAWINGS

The advantages and features provided by one or more embodiments of the invention will become more fully understood from the detailed description given hereinafter and the appended drawings which are given by way of illustration only, and thus are not intended as a definition of the limits of the present disclosure, and wherein:

FIG. 1 is a system configuration diagram of a medical system;

FIG. 2 is a block diagram illustrating a functional configuration of a console;

FIG. 3 is a block diagram illustrating a functional configuration of a diagnostic apparatus;

FIG. 4 is a diagram illustrating a display example of a display screen of a console on which a medical image is displayed;

FIG. 5 is a diagram illustrating the display example of the display screen of the console on which the medical image and first diagnostic assistance information are displayed;

FIG. 6 is a diagram illustrating the display example of the display screen of the console on which the medical image and the second diagnostic assistance information are displayed;

FIG. 7 is a diagram illustrating the display example of the display screen of the console on which the medical image and the second diagnostic assistance information are displayed;

FIG. 8 is a diagram illustrating the display example of the display screen of the diagnostic apparatus in which the medical image on which the first diagnostic assistance information is superimposed and the medical image on which the second diagnostic assistance information is superimposed are displayed in parallel;

FIG. 9 is a diagram illustrating the display example of the display screen of the diagnostic apparatus in which the medical image on which the first diagnostic assistance information and the second diagnostic assistance information are superimposed is displayed;

FIG. 10 is a diagram illustrating an example of the display screen of the diagnostic apparatus in which a manner of displaying the first diagnostic assistance information and the second diagnostic assistance information to be superimposed on the medical image can be selected;

FIG. 11 is a diagram illustrating the display example of the display screen of the diagnostic apparatus when it is selected to superimpose the first diagnostic assistance information on the medical image;

FIG. 12 is a diagram illustrating the display example of the display screen of the diagnostic apparatus in which an overlap region of the first diagnostic assistance information and the second diagnostic assistance information is illustrated on the medical image;

FIG. 13 is a diagram illustrating the display example of the display screen of the diagnostic apparatus in a case of causing a change in the display of the second diagnostic assistance information superimposed on the medical image; and

FIG. 14 is a flowchart illustrating information processing for assisting diagnosis executed by the console and the diagnostic apparatus.

### DETAILED DESCRIPTION

Hereinafter, one or more embodiments of the present disclosure will be described with reference to the drawings. However, the scope of the invention is not limited to the disclosed embodiments.

An embodiment of an information processing apparatus, a diagnostic apparatus, a medical system, and a storage medium according to the present disclosure is described below. However, the scope of the present disclosure is not limited to the illustrated examples.

### Configuration of Medical System

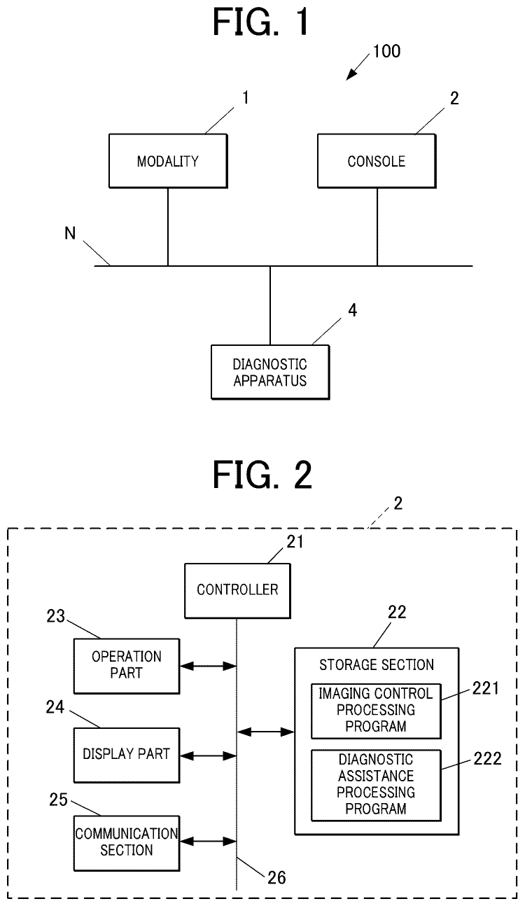

FIG. 1 shows a system configuration of a medical system 100 according to the present embodiment.

As illustrated in FIG. 1, the medical system 100 is configured to include a modality 1, a console 2 which is an information processing apparatus, and a diagnostic apparatus 4. Each apparatus can perform data communication via a communication network N.

In the medical system 100, a medical image captured and generated by the modality 1 can be displayed on the console 2. The apparatuses constituting the medical system 100 are compliant with HL7 (Health Level Seven) or DICOM (Digital Image and Communications in Medicine) standard, and communication between the apparatuses is performed in accordance with HL7 or DICOM. Note that the number and type of modalities 1 and the number of diagnostic apparatuses 4 are not particularly limited.

The modality 1 images a patient (subject) and generates image data of the medical image. The imaging is performed on the basis of examination order information transmitted from a radiology information system (RIS) or the like (not illustrated). As the modality 1, for example, computed radiography (CR), digital radiography (DR), computed tomography (CT), magnetic resonance imaging (MRI), ultra sonography (US), nuclear medicine (NM: nuclear medical examination), endoscopy (ES: endoscope examination), or the like is used. The medical image generated by the modality 1 may be, for example, an X-ray image, a CT image, an MRI image, an ultrasound image, a mammography image, an angiography image, or an endoscopy image.

The modality 1 attaches image attribute information related to the medical image to the medical image. The image attribute information includes a patient ID, a patient name, a date of birth, sex, an imaging date and time, an image ID, a modality, a site, and a direction.

The image data of the medical image generated by the modality 1 can be displayed on a display part 24 or the like of the console 2 to be described later. The image data of the medical image is transmitted to and stored in a medical image management server (not shown). The medical image management server stores and manages the image data of the medical image generated by the modality 1. The medical image management server includes, for example, a picture archiving and communication system (PACS).

### Configuration of Console as Information Processing Apparatus

The console 2 is an imaging control apparatus that controls imaging in the modality 1. The console 2 is operated by a technician such as a medical radiology technician who performs imaging.

The console 2 outputs an imaging condition, an image reading condition, and the like to the modality 1. Furthermore, the image data of the medical image captured by the modality 1 is acquired and appropriately stored in a medical image management server or the like.

Furthermore, according to the present embodiment, the console 2 is an information processing apparatus that attaches diagnostic assistance information to the medical image.

FIG. 2 shows a functional configuration of the console 2.

The console 2 is configured to include a controller 21 (hardware processor), a storage section 22, an operation part 23, a display part 24, a communication section 25, and the like, and each unit is connected by a bus 26.

The controller 21 includes a central processing unit (CPU) and a random access memory (RAM), and comprehensively controls processing operations of sections of the console 2.

The storage section 22 includes, for example, an HDD, a nonvolatile semiconductor memory, or the like. The storage section 22 stores various processing programs, parameters and files necessary for executing the programs, and the like.

The CPU of the controller 21 reads various processing programs stored in the storage section 22, deploys the programs to the RAM, and performs various types of processing in accordance with the programs.

For example, as illustrated in FIG. 2, the storage section 22 according to the present embodiment stores an imaging control processing program 221. First, the CPU of the controller 21 controls capturing of the medical image by the modality 1 in cooperation with the program.

As illustrated in FIG. 2, the storage section 22 according to the present embodiment stores a diagnostic assistance processing program 222. First, in cooperation with the program, the CPU of the controller 21 controls the display part 24 to display at least the medical image generated by the modality 1.

The controller 21 of the present embodiment also functions as a first information generation section (hereinafter simply referred to as generation section) that generates first diagnostic assistance information. The controller 21 as a generation section performs predetermined computer processing on the medical image and generates first diagnostic assistance information. The function as the generation section is implemented by software processing executed by the diagnostic assistance processing program 222 stored in the storage section 22 in cooperation with the CPU of the controller 21. The predetermined computer processing is image diagnosis and image analysis including detection of a lesion location (abnormal location) candidate by computer aided diagnosis (CAD). The image analysis by the CAD is a concept widely including various kinds of processing, and is analysis processing for the medical image using, for example, artificial intelligence (AI) in the present embodiment.

The first diagnostic assistance information is an inference result obtained by computer processing (AI analysis in the present embodiment). By introducing deep learning in the AI analysis, a location having a high degree of likelihood of being a lesion is extracted from an image as a suspected lesion location (abnormal location). The first diagnostic assistance information may include, as reliability, a numerical value or the like indicating the likelihood of being the lesion derived by the AI for each location. The first diagnostic assistance information includes, for example, image information such as an annotation and a mark superimposed on the medical image, and character information such as a comment or a suggestion.

The controller 21 adds, to the first diagnostic assistance information, tag information indicating diagnostic assistance information (CAD information) generated by computer processing.

Furthermore, when the second diagnostic assistance information is input from the operation part 23, the controller 21 according to the present embodiment accepts the input information in cooperation with the diagnostic assistance processing program 222.

The second diagnostic assistance information is input information input by an input operation performed by the technician who operates the console 2. The second diagnostic assistance information is information on the medical image, and is, for example, information on the first diagnostic assistance information derived by AI analysis.

The second diagnostic assistance information includes, for example, image information such as an annotation and a mark superimposed on the medical image, and character information such as a comment and a suggestion.

The controller 21 attaches, to the second diagnostic assistance information, tag information indicating the diagnostic assistance information input by the technician.

Furthermore, in cooperation with the diagnostic assistance processing program 222, the controller 21 according to the present embodiment functions as an output section that controls the communication section 25 to output the diagnostic assistance information to the external device. The “external device” referred to herein is, for example, the diagnostic apparatus 4, a medical image management server (PACS), an image inspection device, or the like which will be described later.

The controller 21 as the output section outputs the first diagnostic assistance information and the second diagnostic assistance information to the external device in a distinguishable manner. The controller 21 as the output section outputs the second diagnostic assistance information in association with the first diagnostic assistance information. The first diagnostic assistance information and the second diagnostic assistance information are output to the external device in association with (linked to) the medical image.

A method of outputting the first diagnostic assistance information and the second diagnostic assistance information in the “distinguishable” manner is not particularly limited. In a case where there are first diagnostic assistance information and second diagnostic assistance information associated with a certain medical image, for example, the controller 21 attaches tag information to each diagnostic assistance information as described above. Thus, it is possible to distinguish whether the diagnostic assistance information is the first diagnostic assistance information or the second diagnostic assistance information. Note that the tag information may include a display form (described later) in which the first diagnostic assistance information and the second diagnostic assistance information are displayed on the display part of the external device.

The operation part 23 includes, for example, a keyboard provided with cursor keys, character/numeral input keys, various function keys, and the like, and a pointing device such as a mouse. The operation part 23 outputs, to the controller 21, an operation signal input by a key operation on the keyboard or a mouse operation. A user (technician) inputs the second diagnostic assistance information by operating the operation part 23. Further, the operation part 23 may be configured by a touch screen laminated on the display part 24. In this case, the operation signal corresponding to the position of the touch operation with the finger or the like of the user (technician) is output to the controller 21.

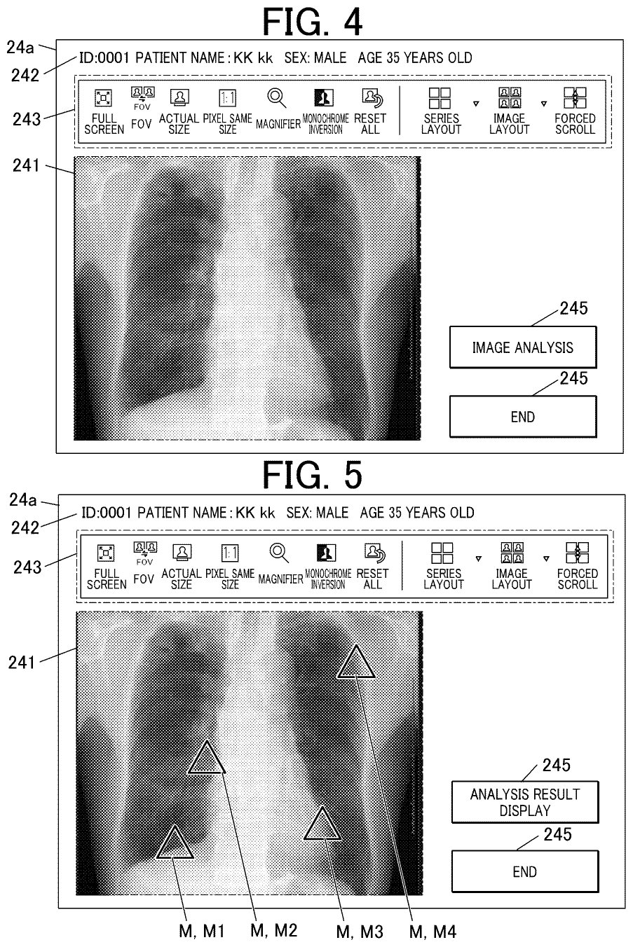

The display part 24 includes a monitor such as a liquid crystal display (LCD). Then, various screens are displayed in accordance with an instruction of a display signal input from the controller 21. FIG. 4 to FIG. 7 show an example of a display screen 24a of the display part 24.

According to the present embodiment, at least a medical image 241 is displayed on the display screen 24a of the display part 24 of the console 2, for example, as illustrated in FIG. 4. In addition, the display screen 24a is provided with an order information field 242 in which information on a patient who is a subject is displayed, a tool display field 243 in which various tools are displayed, and the like. Furthermore, the display screen 24a is provided with various operation buttons 245 to be operated by the user (technician).

The display part 24 may display, for example, as illustrated in FIG. 5, the medical image 241 and the first diagnostic assistance information. In this case, the medical image 241 and the first diagnostic assistance information are displayed on the same screen. FIG. 5 illustrates an example in which triangular marks M1 to M4 are superimposed and displayed on the medical image 241 as the first diagnostic assistance information.

The user (technician) can cause the medical image 241 and the first diagnostic assistance information to be displayed on the same screen of the display part 24 for confirmation. Then, while referring to the medical image 241 and the first diagnostic assistance information, the second diagnostic assistance information such as the mark or the comment is input for the medical image 241 from the operation part 23 or the like.

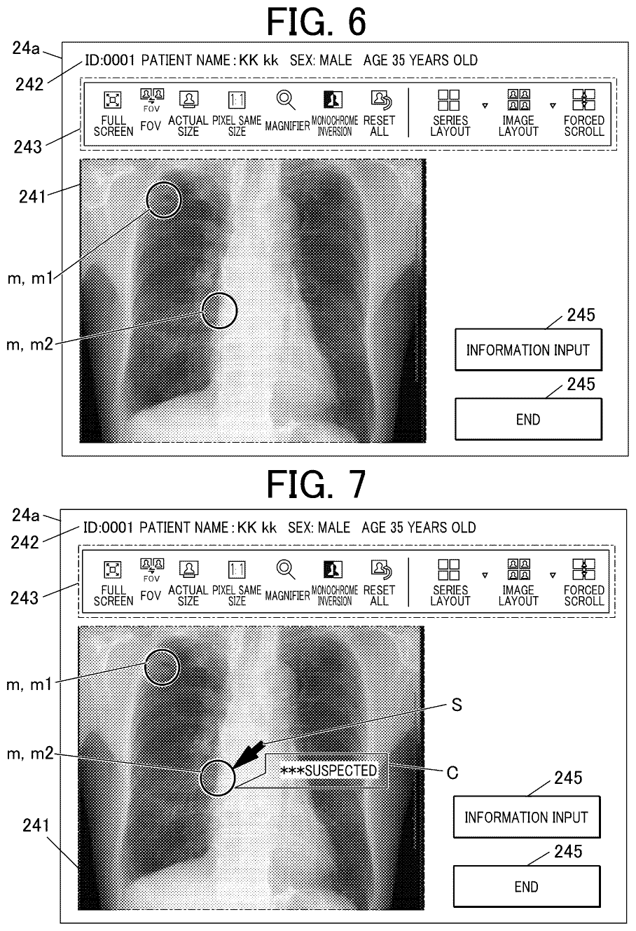

FIG. 6 illustrates an example in which circular marks m1 and m2 are superimposed and displayed on the medical image 241 as the second diagnostic assistance information. In FIG. 7, the marks m1 and m2 and an arrow S indicating a suspected lesion location are superimposed as the second diagnostic assistance information. Furthermore, in the illustrated example, a comment C stating the content of the suspected lesion is superimposed on the medical image 241.

The communication section 25 is constituted by a network interface and the like. The communication section 25 transmits and receives data to and from the external device connected via the communication network N. For example, the communication section 25 receives the medical image obtained by imaging the patient with the modality 1. Furthermore, under the control of the controller 21, the communication section 25 transmits and receives the medical image and an analysis result to and from the external device such as the medical image management server and the diagnostic apparatus 4 as appropriate. That is, the first diagnostic assistance information generated by the controller 21 of the console 2 and the second diagnostic assistance information input by the operation part 23 and accepted by the controller 21 are appropriately transmitted to the external device such as the medical image management server or the diagnostic apparatus 4 via the communication section 25 and stored therein.

### Configuration of Diagnostic Apparatus

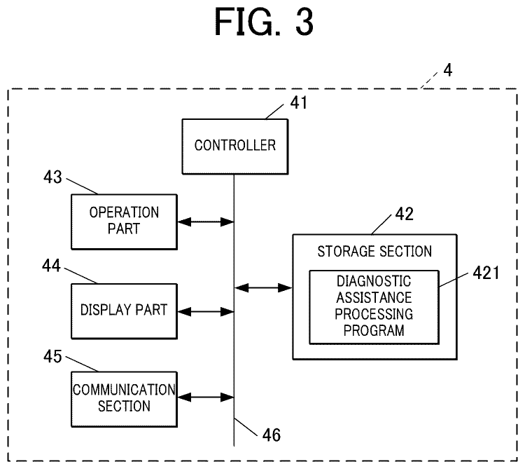

FIG. 3 shows a functional configuration of the diagnostic apparatus 4.

The diagnostic apparatus 4 receives the medical image generated by the modality 1 and the first diagnostic assistance information and the second diagnostic assistance information appended to the medical image in the console 2. The information accepted by the diagnostic apparatus 4 can be referred to by the doctor at the device. The diagnostic apparatus 4 is a terminal device operated by the doctor who is the user. The diagnostic apparatus 4 is, for example, an image reading terminal of a radiologist or a reference terminal referred to by a medical doctor (clinician).

As shown in FIG. 3, the diagnostic apparatus 4 is configured to include a controller 41 (hardware processor), a storage section 42, an operation part 43, a display part 44, a communication section 45, and the like, and each unit is connected by a bus 46.

The controller 41 is composed of a CPU, a RAM and the like, and comprehensively controls the processing operation of each part of the diagnostic apparatus 4. Specifically, the CPU reads various processing programs stored in the storage section 42, deploys the programs to the RAM, and performs various types of processing in accordance with the programs.

The storage section 42 includes an HDD and a nonvolatile semiconductor memory and stores various processing programs and parameters and files necessary for executing the programs.

For example, as illustrated in FIG. 3, the storage section 42 according to the present embodiment stores a diagnostic assistance processing program 421. First, the CPU of the controller 41 functions as an acquisition section that acquires the first diagnostic assistance information and the second diagnostic assistance information in cooperation with the program.

As described above, the first diagnostic assistance information is an inference result of AI analysis generated by performing predetermined computer processing (AI analysis) on the medical image. The second diagnostic assistance information is information that is input with respect to the medical image and can be distinguished from the first diagnostic assistance information.

Further, the controller 41 may function as a third information generation section (hereinafter, simply referred to as a generation section) in cooperation with the diagnostic assistance processing program 421. The controller 41 as the generation section generates third diagnostic assistance information from the first diagnostic assistance information and the second diagnostic assistance information on the basis of a predetermined condition.

Here, the “third diagnostic assistance information” is generated from the first diagnostic assistance information and the second diagnostic assistance information, but is information different from both of the first and second diagnostic assistance information.

For example, in a case where the first diagnostic assistance information and the second diagnostic assistance information are marks having the same color and only shapes are different, the “third diagnostic assistance information” is information adding different colors to the first and second information, respectively. The “third diagnostic assistance information” may be, for example, information in which the first diagnostic assistance information and the second diagnostic assistance information distinguished by different colors or the like are superimposed on one medical image (refer to FIG. 9).

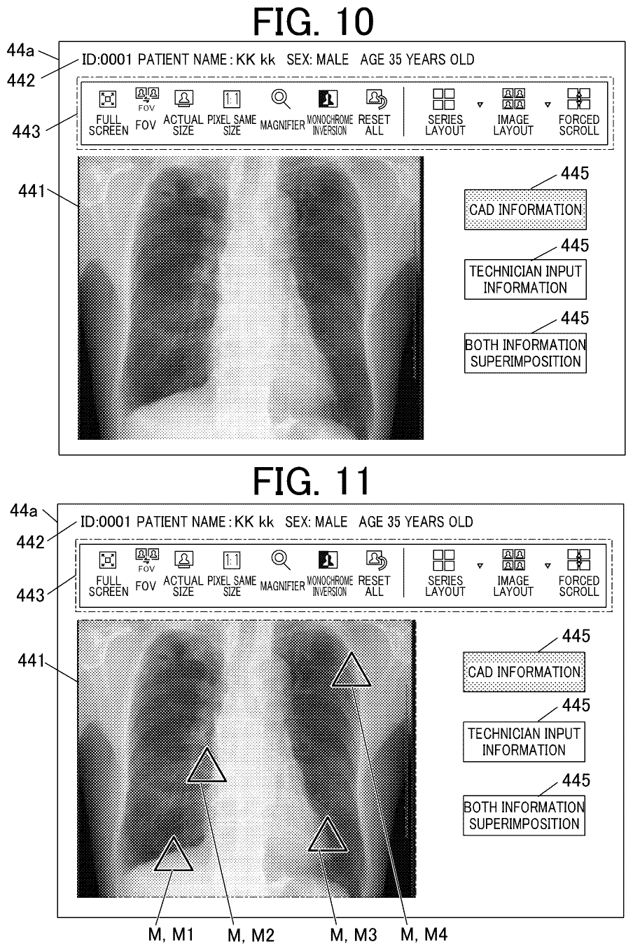

Furthermore, the “third diagnostic assistance information” may be information displayed by, for example, a numeric value indicating a reliability of the likelihood of the lesion derived by the AI. Furthermore, the “third diagnostic assistance information” may show an overlapping area (square area) (see FIG. 10) of the portions detected as the suspicious lesion locations in both the first diagnostic assistance information and the second diagnostic assistance information, or the like. The degree of overlap, which indicates how much the parts detected as the suspected lesion locations in the first and second diagnostic assistance information overlap with each other, is calculated by, for example, the controller 41 as the generation section.

The image to be displayed on the display part 44, which will be described later, may not be the first diagnostic assistance information or the second diagnostic assistance information itself, but may be “third diagnostic assistance information” or the like generated based on the first diagnostic assistance information and the second diagnostic assistance information.

The operation part 43 includes, for example, the keyboard provided with the cursor keys, the character/numeral input keys, various function keys, and the like, and the pointing device such as the mouse. The operation part 43 outputs, to the controller 41, the operation signal input by the key operation on the keyboard or the mouse operation. Further, the operation part 43 may be configured by a touch screen laminated on the display part 44. In this case, the operation signal corresponding to the position of the touch operation with the finger or the like of the user (technician) is output to the controller 41.

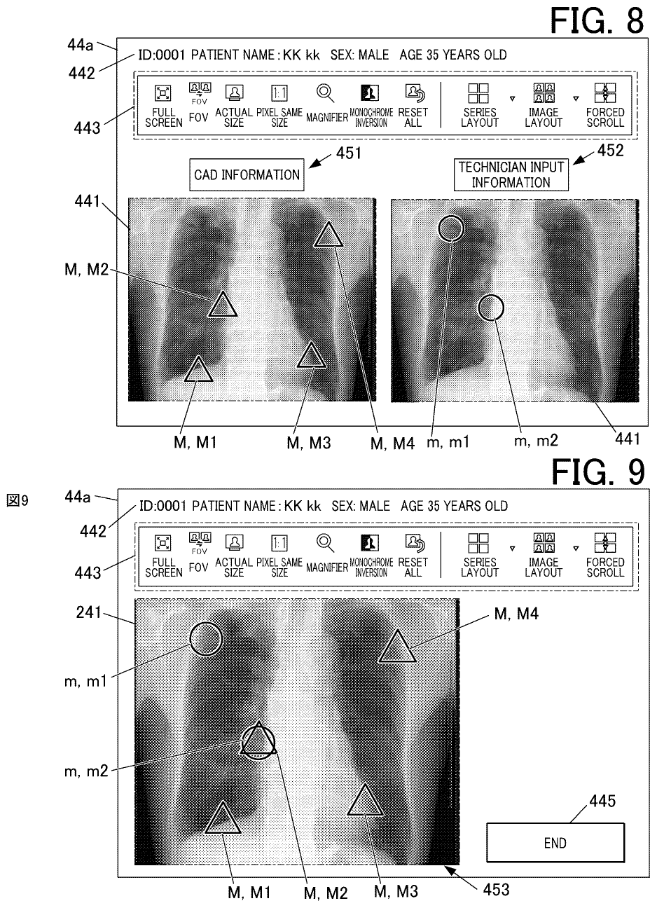

The display part 44 includes a monitor such as a liquid crystal display (LCD) and displays various screens according to the instruction of a display signal input from the controller 41. FIG. 8 to FIG. 11 show examples of the display screen 44a of the display part 44.

According to the present embodiment, the display part 44 of the diagnostic apparatus 4 is a “second display part” that displays at least the medical image 441 (see FIG. 10). The display part 44 may display the medical image 441 and the first diagnostic assistance information (see FIG. 11). In this case, it is preferable to display the medical image 441 and the first diagnostic assistance information on the same screen. Furthermore, as illustrated in FIG. 8, the medical image 441, the first diagnostic assistance information, and the second diagnostic assistance information may be displayed on the same screen.

According to the present embodiment, under the control of the controller 41, the display part 44 displays the first diagnostic assistance information and the second diagnostic assistance information in the distinguishable manner.

Here, the “distinguishable” display is, for example, display in a different display manner. It is sufficient that the two information can be distinguished from each other, and specifically, how the two pieces of information are displayed in different display manners is not particularly limited.

For example, in FIG. 8, the image in which the first diagnostic assistance information is superimposed on the medical image 441 and the image in which the second diagnostic assistance information is superimposed on the medical image 441 are displayed on the same screen. Note that in FIG. 8 and the like and the following description, the first diagnostic assistance information is also referred to as “CAD information”. The second diagnostic assistance information is also referred to as “technician input information”.

As described above, the first diagnostic assistance information and the second diagnostic assistance information output from the console 2 are associated with the medical image. Furthermore, the tag information indicating whether the information is the first or second diagnostic assistance information is appended to each piece of information by the controller 21 of the console 2.

The controller 41 displays, in a CAD information field 451, the image on which the first diagnostic assistance information is superimposed on the basis of the tag information (refer to FIG. 8). The image on which the second diagnostic assistance information is superimposed is displayed in a technician input information field 452. Thus, even if the first and second diagnostic assistance information are displayed in parallel on the same display screen 44a, the user (doctor) does not misunderstand or become confused.

Displaying in the “distinguishable” manner includes displaying with at least one of a character, a color, a size, a font, and a shape being changed, for example. More specifically, for example, this includes changing the color, size, shape, and the like of the mark or the like between the first diagnostic assistance information and the second diagnostic assistance information. Furthermore, this includes changing the color, size, and font of letters of text information such as a comment and a suggestion between the first diagnostic assistance information and the second diagnostic assistance information.

Furthermore, the display part 44 may display either the first diagnostic assistance information or the second diagnostic assistance information on the basis of a certain condition (predetermined condition). For example, the instruction to request to refer to the first diagnostic assistance information may be input from the operation part 43 of the diagnostic apparatus 4. In this case, the controller 41 allows the display part 44 to display, for example, only the first diagnostic assistance information superimposed on the medical image 441. Furthermore, for example, the instruction to request to refer to the second diagnostic assistance information may be input from the operation part 43 of the diagnostic apparatus 4. In this case, the controller 41 allows the display part 44 to display, for example, only the second diagnostic assistance information superimposed on the medical image 441. The information can be emphasized by superimposing and displaying either one of them on the medical image 441.

For example, as illustrated in FIG. 10, a tab or a button 445 for the first diagnostic assistance information and the tab or the button 445 for the second diagnostic assistance information may be provided on the display screen of the display part 44. In this case, when the user (doctor) selects any of the tabs or the button 445 with the pointing device such as the mouse, the selected information may be superimposed and displayed on the medical image 441. In addition, both information superimposition button for giving an instruction to superimpose and display both of the first and second diagnostic assistance information may be provided.

The example illustrated in FIG. 10 illustrates the example in which the CAD information is selected. In this case, as shown in FIG. 11, only the CAD information is superimposed on the medical image 441 and displayed on the display screen 44a. When the user (doctor) selects superimposition of both information, for example, as shown in FIG. 9, both the first and second diagnostic assistance information are superimposed and displayed on the medical image 441. Even when two information are superimposed as described above, the shapes, colors, and the like of the marks indicating the respective information are different in the present embodiment. Therefore, the user (doctor) can correctly distinguish between the two information.

Furthermore, the display part 44 may display one medical image 441 in which both the first diagnostic assistance information and the second diagnostic assistance information are superimposed on the medical image 441. In this case, as illustrated in FIG. 9, the first diagnostic assistance information and the second diagnostic assistance information are expressed on one medical image 441. Thus, the user (doctor) can confirm both information without switching pages or the like.

Furthermore, the display part 44 may display the first diagnostic assistance information and the second diagnostic assistance information so that the difference is distinguishable. For example, a portion common to the first diagnostic assistance information and the second diagnostic assistance information is set to be black. Further, information in only the first diagnostic assistance information is set to green, and information in only the second diagnostic assistance information is set to red. Thus, the common portion and the different portion of the two diagnostic assistance information can be presented to the user (doctor) in a manner that is easy to understand.

For example, in the case of the above example, the mark M2 and the mark m2 in FIG. 9 are black. Further, the marks M1, M3, and M4 are green, and the mark m1 is red.

In some cases, the technician is not concerned about the diagnostic assistance information by the CAD (AI) such as the AI analysis. In addition, the technician may be concerned about a portion different from the determination by the AI. There may be a case in which there is interest in whether the technician is concerned or not about the diagnostic assistance information by the CAD (AI) or whether the technician is concerned about a location different from the CAD (AI). Therefore, the difference between the first diagnostic assistance information and the second diagnostic assistance information is displayed in the distinguishable manner. As a result, the user (doctor) can easily grasp the difference between the determination by the CAD (AI) and the determination by the technician.

For example, in the example illustrated in FIG. 9, the mark M2 and the mark m2 are commonly extracted as suspected lesion locations. In addition, it is understood that the technician is not concerned about the marks M1, M3, and M4. On the other hand, it is found that although the mark m1 is not detected by the AI analysis, the technician considers the mark m1 as the suspected lesion location. By superimposing and displaying the first and second diagnostic assistance information on the one medical image 441, the user (doctor) can easily confirm the difference or common points between the first and second diagnostic assistance information.

The controller 41 as the generation section may generate the third diagnostic assistance information from the first diagnostic assistance information and the second diagnostic assistance information. In this case, the third diagnostic assistance information may be displayed on the display screen 44a of the display part 44. In this case, the first diagnostic assistance information, the second diagnostic assistance information, and the third diagnostic assistance information may be displayable while being appropriately switched by the user (doctor) operation.

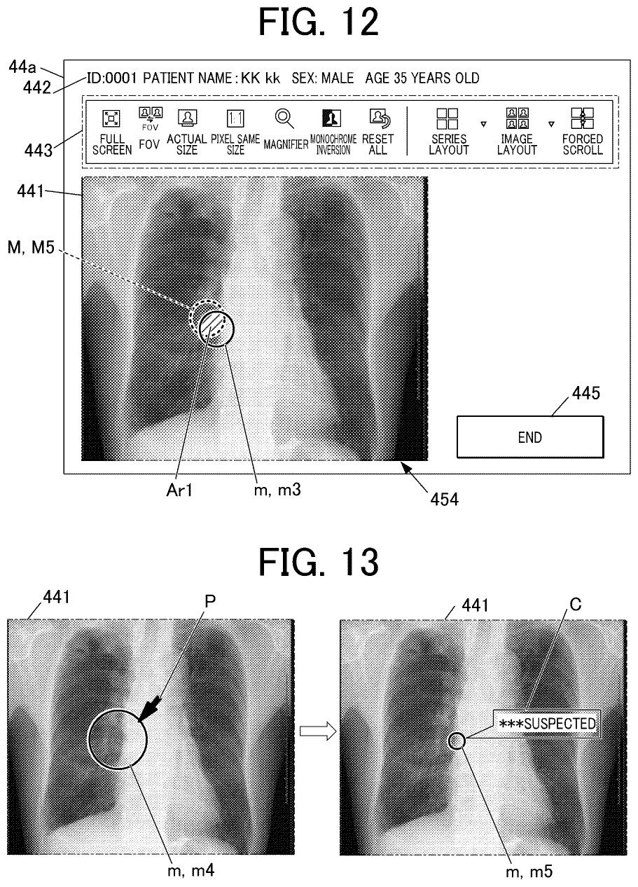

For example, in a case where the first and second diagnostic assistance information indicate substantially the same position, as illustrated in FIG. 12, the overlap of the first and second diagnostic assistance information may be displayed on the display screen 44a.

For example, in FIG. 12, the mark M5 that is the first diagnostic assistance information (CAD information) is indicated by a broken line. The mark m3, which is the second diagnostic assistance information (technician input information), is indicated by a solid line. The positions of the mark M5 and the mark m3 are slightly shifted from each other, and in FIG. 12, an overlap region Ar1 in which the marks overlap each other is indicated by hatching.

As illustrated in FIG. 12, in a case where positions of the first and second diagnostic assistance information are shifted from each other or in a case where only a part of the first and second diagnostic assistance information overlap with each other, a range, area, and the like of the overlap region Ar1 may be indicated as the third diagnostic assistance information. The range, the area, and the like of the overlap region Ar1 are calculated by the controller 41 serving as the generation section.

Furthermore, as illustrated in FIG. 8, the first diagnostic assistance information and the second diagnostic assistance information may be displayed side by side on the display screen 44a of the display part 44. In this case, one of the above may be displayed on the entire screen by the user (doctor) operation. Furthermore, the medical image on which the first diagnostic assistance information is superimposed and the medical image on which the second diagnostic assistance information is superimposed may be appropriately switched and displayed.

The display switching operation is performed by, for example, a scroll operation or a click (selection) operation. When a mouse is used as the operation part 43, the screen may be switched by operating a scroll wheel or a mouse wheel.

In addition, one image selected by the user (doctor) may be displayed on the entire display screen 44a, and the other images may be displayed to be superimposed on a lower portion, a side portion, or the like of the display screen 44a (for example, stack display). In the case of the stack display, when the image displayed in an overlapping manner on the lower portion of the display screen 44a or the like is selected by a tap operation or the like, the image may be displayed on the entire display screen 44a.

Furthermore, for example, on the display screen 24a (see FIG. 6 and FIG. 7) of the console 2, the user (technician) may widely surround the location suspected to be the lesion with the circle mark m4. Then, the mark m5, a comment C, or the like indicating a location to be particularly noted may be attached to a certain point (range) in the image.

In this case, a moving (changing) display as illustrated in FIG. 13 may be provided on the display part 44 of the diagnostic apparatus 4. That is, as illustrated on the left side of FIG. 13, when the user (doctor) brings the pointer of the mouse, the cursor, or the like close to the mark m4, the mark m4 may be reduced to the mark m5 which is the location of interest as illustrated on the right side of the drawing. At this time, the comment C or the like may appear on the display screen 44a. Performing the display with such movement (operation) can indicate the location that should be particularly noted to the user (doctor) in a manner that is easy to understand.

Furthermore, for example, the first and second diagnostic assistance information may be presented to the user (doctor) by a tooltip method or the like. In this case, for example, when the user (doctor) moves the pointer of the mouse, the cursor, or the like close to the circular marks m1 to m5 and the like, a comment, a supplementary explanation, and the like are displayed around the marks.

The display mechanism and specifications realized in the display part 44 of the diagnostic apparatus 4 are set in the console 2. Specifically, for example, it is conceivable to set these display aspects and the like in the tag information or the like attached to the first and second diagnostic assistance information.

The communication section 45 of the diagnostic apparatus 4 is configured by the network interface or the like, and can transmit and receive information to and from various devices connected via the communication network N. The apparatus with which the diagnostic apparatus 4 communicates via the communication section 45 is, for example, the console 2 or the medical image management server (PACS). The diagnostic apparatus 4 can receive transmission of the medical image and the diagnostic assistance information from the console 2, the medical image management server (PACS), or the like via the communication section 45.

### Operation in Medical System

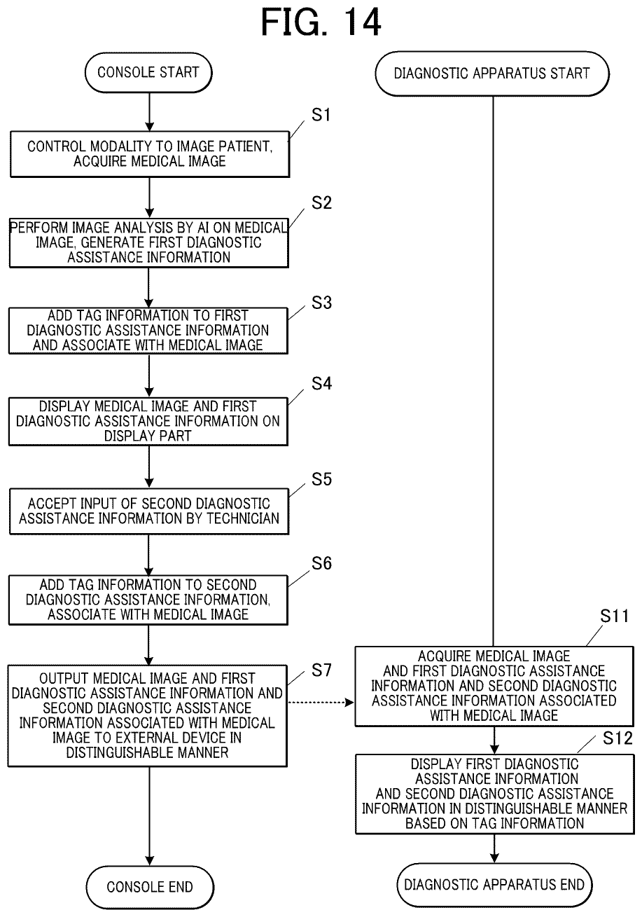

Next, the operation of the medical system 100 will be described with reference to FIG. 14.

FIG. 14 is a flowchart illustrating the processing by the console 2 that is the information processing apparatus and the diagnostic apparatus 4 configuring the medical system 100 of the present embodiment.

As illustrated in FIG. 14, first, when an imaging order is input from the doctor, the console 2 controls a corresponding modality in accordance with the order to cause the modality to image the patient (subject). Then, the console 2 acquires the captured medical image (step S1). The acquired medical image may be displayed on the display part 24 of the console 2.

As illustrated in FIG. 4, the medical image 241, the order information display field 242, the tool display field 243, various operation buttons 245, and the like are provided in the display screen 24a of the display part 24. For example, in FIG. 4, an image analysis button for instructing the image analysis by the computer processing, an end button to be operated when ending processing, and the like are provided as the operation button 245. The configuration, display content, and the like of the display screen 24a are not limited to the illustrated example.

When the medical image is acquired and the image analysis button is operated by the user (technician) operating the console 2, the controller 21 as the generation section performs the image analysis on the medical image by the AI and generates the first diagnostic assistance information (step S2). Even if the user (technician) does not perform the button operation or the like, when the medical image is acquired, the controller 21 may automatically perform the image analysis by the AI.

The controller 21 attaches, to the first diagnostic assistance information, the tag information indicating that the information is CAD information that is a result of computer processing (AI analysis). Then, the first diagnostic assistance information is associated with the medical image (step S3). The medical image and the first diagnostic assistance information associated therewith are stored in the storage section 22 or the like.

Upon completion of the image analysis by the AI, the controller 21 allows the display part 24 to display the medical image and the first diagnostic assistance information (step S4). To be specific, as illustrated in FIG. 5, the display screen 24a in which the image of the first diagnostic assistance information is superimposed on the medical image 241 is displayed on the display part 24. Note that in FIG. 5, an analysis result display button is provided as the operation button 245 on the display screen 24a. The analysis result may be automatically displayed on the display screen 24a even if the operation button 245 is not operated.

In the example illustrated in FIG. 5, the first diagnostic assistance information indicates that a triangular mark is given to the suspected lesion location. In the illustrated example, four suspected lesion locations with the marks M1 to M4 are detected by the AI. The user (technician) can check the first diagnostic assistance information, which is the CAD information, on the display part 24. The user (technician) inputs his/her opinion on the medical image and the first diagnostic assistance information from the operation part 23 or the like (see FIG. 6).

The opinion input by the user (technician) is accepted as the second diagnostic assistance information by the controller 21 (step S5).

In the example illustrated in FIG. 6, the second diagnostic assistance information indicates that the suspected lesion location is marked with a circle mark. In the illustrated example, the two suspected lesion locations of the marks m1 and m2 are pointed out by the user (technician).

Note that the analysis result by the AI and the input result by the technician may be reflected as data used for subsequent machine learning.

The controller 21 attaches, to the second diagnostic assistance information, the tag information indicating that the information is the opinion of the technician. Then, the second diagnostic assistance information is associated with the medical image and the first diagnostic assistance information (step S6). The medical image and the first diagnostic assistance information associated therewith may be stored in the storage section 22 or the like.

The tag information attached to the first diagnostic assistance information and the second diagnostic assistance information includes the following. That is, display correspondences when the first diagnostic assistance information and the second diagnostic assistance information are displayed are respectively corresponded.

Then, the controller 21 identifiably outputs the medical image and the first diagnostic assistance information and the second diagnostic assistance information associated with the medical image to the external device (step S7).

According to the present embodiment, the information output from the console 2 is acquired by the diagnostic apparatus 4 as the external device (step S11). Output from the console 2 are the medical image and the first diagnostic assistance information and the second diagnostic assistance information associated with the medical image. Note that the medical image and the diagnostic assistance information are not necessarily directly output from the console 2 to the diagnostic apparatus 4. For example, the medical image and the diagnostic assistance information may be output from the console 2 to the medical image management server that is the external device. In this case, the diagnostic apparatus 4 may acquire the medical image and the diagnostic assistance information from the medical image management server. In this case, the diagnostic apparatus 4 transmits an acquisition request for the medical image or the like to the medical image management server via the communication section 45. Thus, the medical image and the information associated therewith are transmitted from the medical image management server to the diagnostic apparatus 4.

The controller 41 of the diagnostic apparatus 4 acquires the medical image and the first diagnostic assistance information and the second diagnostic assistance information associated with the medical image. Then, based on the tag information, the controller 41 allows the display part 44 to display the first diagnostic assistance information and the second diagnostic assistance information in the distinguishable manner (step S12).

According to the present embodiment, for example, as shown in FIG. 8 and the like, the first diagnostic assistance information and the second diagnostic assistance information are different from each other in the shape, color, and the like of the mark, and can be distinguished from each other. Therefore, even in a case where one medical image is accompanied by the first and second diagnostic assistance information, the user (doctor) who sees the image can easily determine whether the information is the first and second diagnostic assistance information, respectively. Note that the first diagnostic assistance information and the second diagnostic assistance information are not limited to the illustrated example as long as they can be distinguished from each other.

As described above, the information processing apparatus according to the present embodiment is the console 2 including the controller 21 that controls the capturing of the medical image by the modality 1. The console 2 includes at least the display part 24 for displaying the medical image and the operation part 23 as the input section for inputting the second diagnostic assistance information regarding the medical image. The controller 21 also functions as the first information generation section (generation section) that generates the first diagnostic assistance information by performing predetermined computer processing (e.g., AI processing) on the medical image. Furthermore, the controller 21 also functions as the output section that outputs the first diagnostic assistance information and the second diagnostic assistance information to the external device in a distinguishable manner.

There are cases in which the CAD information generated by AI processing and the technician input information input by the technician are attached to one medical image. Depending on the content or manner of the diagnostic assistance information, it may be difficult to determine whether the diagnostic assistance information is the CAD information or the technician input information.

For example, the capability of the technician is high in an ultrasound image, a mammography image, or the like. For this reason, a manner is conceivable in which the technician checks whether or not the result of the computer processing (for example, AI processing) is appropriate. In the case of such an manner, it is desired that the doctor can identify which is the technician input information with higher reliability.

Furthermore, the technician may not be concerned about the CAD information or the technician may be concerned about another location. If the CAD information and the technician input information cannot be distinguished from each other, the doctor cannot reflect the judgment of the technician in the diagnosis or the like.

Turning to the present embodiment, both information are distinguishably output from the console 2 to the diagnostic apparatus 4 or the like. Therefore, the doctor who sees the diagnostic assistance information can determine whether the diagnostic assistance information is the CAD information or the technician input information without misunderstanding. Thus, a correct diagnosis or the like can be derived from the diagnostic assistance information.

The display part 24 of the console 2 displays the medical image and the first diagnostic assistance information on the same screen. Therefore, the technician can input the second diagnostic assistance information while checking the first diagnostic assistance information.

Furthermore, the diagnostic apparatus 4 that has acquired the CAD information and the technician input information causes the display part 44 to display the information in a manner such that it can be distinguished whether the information is the CAD information or the technician input information. Therefore, the doctor who makes a diagnosis or the like can correctly determine the diagnostic assistance information and make use of the information for the diagnosis or the like.

The first diagnostic assistance information and the second diagnostic assistance information may be displayed side by side on one screen, or may be switched and displayed. In this case, a common point and a different point can be easily confirmed by comparing both of them.

The first diagnostic assistance information and the second diagnostic assistance information may be superimposed on one medical image. Also in this case, both can be easily compared with each other, which can be useful for the diagnosis or the like.

Furthermore, the diagnostic apparatus 4 may generate the third diagnostic assistance information from the first diagnostic assistance information and the second diagnostic assistance information. In this case, the display part 44 may display the third diagnostic assistance information. As a result, more specific information can be provided to the doctor who performs the diagnosis or the like, and the amount of information for determination at the time of diagnosis or the like increases.

Note that the descriptions in the above embodiment are examples of the information processing apparatus, the diagnostic apparatus, the medical system, the program, and the information processing method according to the present disclosure, and the present disclosure is not limited thereto. The detailed configuration and detailed operation of each device constituting the system can also be appropriately changed without departing from the scope of the present disclosure.

For example, in the above embodiment, the controller 21 of the console 2, which is the information processing apparatus, obtains the first diagnostic assistance information, which is the CAD information, by the analysis using the AI. However, the predetermined computer processing for generating the first diagnostic assistance information (diagnostic assistance information by CAD) is not limited to the AI analysis.

For example, the generation of the diagnostic assistance information by the CAD may be performed by a rule-base algorithm based on a difference in density, a feature of a shape, or the like.

In this case, the suspected lesion location (abnormal location) is detected according to the rule based on the specific pattern or standard. For example, the abnormality is detected on the basis of physical features such as difference in gradation and shape in the image, detection of edges, abnormality in size, positional relationship, and the like.

The generation of the diagnostic assistance information by the CAD may be performed by threshold value processing.

In this case, the location whose brightness or density exceeds a certain standard by threshold value setting is detected as the suspected lesion location (abnormal location). For example, it is conceivable that a nodule is determined to be the abnormality when the nodule exceeds a certain size.

Furthermore, the generation of the diagnostic assistance information by the CAD may be performed by shape analysis and a basic statistical method.

In this case, through simple statistical analysis, the difference in shape or density is used to grasp the features. Thus, the suspected lesion location (abnormal location) is easily specified.

The processing without using the AI as described above has the following advantages.

First, in a case where the AI is not used, calculation resources and the time required for the image processing are small. Therefore, it is possible to efficiently perform processing.

In addition, since the standard and rules of the processing are clear, the result is easy to understand and the doctor tends to interpret easily.

On the other hand, processing without using the AI has the following limitations.

That is, there is no ability to recognize a complicated pattern as in deep learning in the AI. Therefore, the possibility of oversight or erroneous detection increases.

In addition, since rules and standards are fixed, there is a limit to the detection of new symptoms and minute abnormality.

However, processing that does not use the AI, such as the above-described rule-base method, is still useful for diagnostic assistance or for use under specific conditions. Therefore, the present disclosure does not exclude these from the predetermined computer processing of generating the first diagnostic assistance information.

Furthermore, although the example in which a chest image is used as the medical image has been illustrated in the above-described embodiment, the imaging region is not limited to the chest. Furthermore, the medical image to be processed may be a plurality of slice images obtained by CT or the like, a multi-frame image (moving image), or the image obtained by converting the image obtained by CT, MRI, or the like into 3D.

Furthermore, the program for executing each process in each apparatus may be stored in a portable recording medium. In addition, a carrier wave may be applied as a medium for providing program data via the communication line.

Although embodiments of the present disclosure have been described and illustrated in detail, the disclosed embodiments are made for purposes of illustration and example only and not limitation. The scope of the present disclosure should be interpreted by terms of the appended claims.

What is claimed is:

1. An information processing apparatus comprising:
a first hardware processor that controls imaging of a medical image and that generates first diagnostic assistance information by performing predetermined computer processing on the medical image;
a first display that displays at least the medical image; and
an inputter for inputting second diagnostic assistance information regarding the medical image,
wherein the hardware processor outputs the first diagnostic assistance information and the second diagnostic assistance information to an external device in a distinguishable manner.

2. The information processing apparatus according to claim 1, wherein,
the first display displays the medical image and the first diagnostic assistance information on a same screen, and
the second diagnostic assistance information is information regarding the first diagnostic assistance information.

3. A diagnostic apparatus comprising:
a second hardware processor that acquires first diagnostic assistance information generated by performing predetermined computer processing on a medical image, and second diagnostic assistance information that is information input regarding the medical image and that is information distinguishable from the first diagnostic assistance information; and
a second display that displays at least the medical image.

4. The diagnostic apparatus according to claim 3, wherein the second display displays the first diagnostic assistance information and the second diagnostic assistance information in a distinguishable manner.

5. The diagnostic apparatus according to claim 3, wherein the second display causes one of the first diagnostic assistance information and the second diagnostic assistance information to be displayed based on a predetermined condition.

6. The diagnostic apparatus according to claim 3, wherein,
the second hardware processor generates third diagnostic assistance information from the first diagnostic assistance information and the second diagnostic assistance information based on a predetermined condition, and
the second display displays the third diagnostic assistance information generated by the second hardware processor.

7. The diagnostic apparatus according to claim 3, wherein the second display displays a difference between the first diagnostic assistance information and the second diagnostic assistance information in a distinguishable manner.

8. The diagnostic apparatus according to claim 4, wherein the second display causes the first diagnostic assistance information and the second diagnostic assistance information to be displayed side by side on a display screen.

9. The diagnostic apparatus according to claim 4, wherein the second display displays the medical image in which the first diagnostic assistance information is superimposed on the medical image and the medical image in which the second diagnostic assistance information is superimposed on the medical image side by side or in a switching manner.

10. The diagnostic apparatus according to claim 4, wherein the second display displays one medical image in which the first diagnostic assistance information and the second diagnostic assistance information are superimposed on the medical image.

11. The diagnostic apparatus according to claim 4, wherein the second display displays the first diagnostic assistance information and the second diagnostic assistance information in the distinguishable manner by changing at least one of a character, a color, a size, a font, and a shape of the first diagnostic assistance information and the second diagnostic assistance information.

12. A medical system including the information processing apparatus according to claim 1, the medical system comprising:
a diagnostic apparatus including, a second hardware processor that acquires the first diagnostic assistance information and the second diagnostic assistance information, and a second display that displays at least the medical image.

13. The medical system according to claim 12, wherein the second display further displays the first diagnostic assistance information and the second diagnostic assistance information in a distinguishable manner.

14. The medical system according to claim 12, wherein the second display displays one of the first diagnostic assistance information and the second diagnostic assistance information based on a predetermined condition.

15. The medical system according to claim 12, wherein the second display displays third diagnostic assistance information derived from the first diagnostic assistance information and the second diagnostic assistance information based on a predetermined condition.

16. A non-transitory computer readable storage medium storing a program that causes a computer of an apparatus including a display to,
control capturing of a medical image,
cause the display to display at least the medical image,
generate first diagnostic assistance information by performing predetermined computer processing on the medical image,
accept input of second diagnostic assistance information on the medical image, and
output the first diagnostic assistance information and the second diagnostic assistance information to the external device in a distinguishable manner.
